(19) United States
(12) Patent Application Publication
(10) Pub. No.: US 2026/0183522 A1
(43) Pub. Date: Jul. 2, 2026

(54) CATHETER AND METHODS OF USING SAME

(71) Applicant: The United States Government as represented by the Department of Veterans Affairs, Washington, DC (US)

(72) Inventor: Muta M. Issa, Decatur, GA (US)

(21) Appl. No.: 19/129,959

(22) PCT Filed: Oct. 18, 2023
(86) PCT No.: PCT/US2023/077147

§ 371 Date: May 14, 2025

Related U.S. Application Data
(60) Provisional application No. 63/426,588, filed on Nov. 18, 2022.
(60) Provisional application No. 63/483,333, filed on Feb. 6, 2023.

(51) Int. Cl. A61M 29/02; A61M 25/09; A61M 25/10

(52) U.S. Cl. CPC A61M 29/02; A61M 25/10; A61M 25/09; A61M 2025/1061; A61M 2029/025

(57) ABSTRACT

A catheter includes a conduit assembly. The conduit assembly can have a conduit having a proximal end portion, a distal end portion, and a lumen that extends through the conduit from the proximal end portion to the distal end portion. A balloon can be coupled to and configured to extend radially outwardly of the conduit. The distal end portion of the conduit has a distal tip of the conduit. At least one inlet to the lumen is positioned between the balloon and the distal tip of the conduit. A guidewire dilator is coupled to the conduit.

### CROSS-REFERENCE TO RELATED APPLICATION

This application claims priority to and the benefit of the filing date of U.S. Provisional Patent Application Nos. 63/426,588, filed Nov. 18, 2022, and 63/483,333, filed Feb. 6, 2023, each of which is hereby incorporated herein by reference in its entirety.

### FIELD

This application relates to catheters and methods of placing such catheters within a subject. For example, the catheters disclosed herein can be wire-guided dilation catheters.

### BACKGROUND

Conventionally, placement of bladder catheters is currently blind and unguided. However, blind, unguided placement can lead to failure to access/enter the bladder, resulting in traumatic urethral catheterization (TUC). Incidence of TUC is 13.4 per 1,000 catheters and is a cause of significant short-term and long-term morbidity. Contributing factors include: the blind nature of the procedure, the high relevance of prostate enlargement/prostatic urethral narrowing, natural anatomical curvatures and tortuosity of the urethra, and narrowing of the urethra related to prior instrumentation/procedure or strictures. Consequences include injury to urethra resulting in bleeding, false passage, failure to empty the bladder, urinary retention, pain, suffering, delays and cost. Other consequences can include a need for cystourethroscopy to gain access into the bladder, prolonged Foley catheterization, placement of supra-pubic tube in selective cases, increased risk of infection and sepsis, hospitalization, long-term damage to the urethra with development of urethral stricture requiring further management, procedures, etc., delay and/or cancellation of a procedure (in operating room cases), significant cause of short-term and long-term morbidity, and significant cost. In one series of 37 iatrogenic ureteral injuries during 6-month period, the average cost incurred in managing a single traumatic urethral catheterization was $10,048.

More generally, it can be necessary to place catheters in various lumens in the body. Various geometries, such as strictures and turns can make traversal difficult. Accordingly, an improved way of placing catheters is desirable.

### SUMMARY

Disclosed herein, in one aspect, is a catheter comprising a conduit assembly. The conduit assembly can have a conduit having a proximal end portion, a body, a distal end portion, and a lumen that extends through the conduit from the proximal end portion to the distal end portion. A balloon can be coupled to and configured to extend radially outwardly of the conduit. The distal end portion of the conduit can have a distal tip of the conduit and at least one inlet/outlet to the lumen that is positioned between the inflatable balloon and the distal tip of the conduit. A guidewire is coupled to the conduit. The guidewire can extend distally of the distal end portion of the conduit.

A system can comprise a catheter as disclosed herein. The catheter can comprise a fluid inlet in fluid communication with the balloon. The fluid inlet can be configured to receive a fluid to inflate the balloon. A fluid source can be coupled to the fluid inlet of the catheter.

In one aspect, a method can comprise inserting the distal end portion of the conduit of a catheter as disclosed herein through the urethra of a patient to access/enter into the urinary bladder. The balloon can be inflated to secure the distal end of the conduit within the bladder.

In one aspect, a catheter comprises a conduit having a proximal end portion, a distal end portion, and a lumen that extends through the conduit from the proximal end portion to the distal end portion. The distal end portion of the conduit has a distal tip of the conduit and at least one inlet to the lumen and a guidewire is coupled to the conduit. The guidewire extends distally of the distal end portion of the conduit.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is side view of a distal end portion of a catheter as disclosed herein, with a balloon of the catheter in a deflated configuration.

FIG. 2 is a side view of the portion of the catheter of FIG. 1, with a balloon of the catheter in an inflated configuration.

FIG. 3 is a side view of the portion of the catheter of FIG. 1, with the guidewire of the catheter in a slightly flexed position.

FIG. 4 is a side view of the portion of the catheter of FIG. 1, with the guidewire of the catheter in a more flexed position.

FIG. 5A is a schematic view of the catheter as disclosed herein. FIG. 5B is a cross section of a portion of a catheter as disclosed herein.

FIG. 6 is a schematic view of a catheter as disclosed herein.

FIG. 7A shows a side view of a proximal portion of a catheter as disclosed herein. FIG. 7B shows a side view of the catheter with the guidewire in a first looped configuration. FIG. 7C shows a side view of the catheter with the guidewire in a second looped configuration.

FIG. 8A shows a side view of a proximal portion of a catheter as disclosed herein and a connecting tube being received within the proximal portion of the catheter. FIG. 8B shows a side view of a proximal portion of a catheter as disclosed herein and the connecting tube received within the proximal portion of the catheter.

FIG. 9 shows a side view of a proximal portion of a catheter as disclosed herein.

FIG. 10 shows a side view of a proximal portion of a catheter as disclosed herein with a plug received therein and retaining the guidewire relative to the catheter in one aspect.

FIG. 11 shows a side view of a proximal portion of a catheter as disclosed herein with a plug received therein and retaining the guidewire relative to the catheter in another aspect.

FIG. 12 shows a side view of a proximal portion of a catheter as disclosed herein with a plug received therein and retaining the guidewire relative to the catheter in another aspect.

FIG. 13 shows a side view of a proximal portion of a catheter as disclosed herein with an anchor coupled to the proximal portion of the conduit.

FIG. 14A is a side view of a catheter as disclosed herein with a guidewire being coupled to a body of a dilator in the undeployed position, i.e., within the lumen of the catheter. FIG. 14B is a side view of the catheter of FIG. 14A, with the dilator and guidewire in another position relative to the conduit, i.e., deployed out of the distal tend of the catheter.

FIG. 15A is a schematic side view of a portion of a catheter as disclosed herein. FIG. 15B is a schematic side view of the portion of the catheter, showing the dilator of the catheter flexing.

FIG. 16A is a schematic side view of a portion of a catheter as disclosed herein, wherein the catheter has a dilator defined by the distal end portion of the conduit. FIG. 16B is a schematic side view of the portion of the catheter, showing the dilator of the catheter flexing.

FIGS. 17A-17E show side views of different guidewires and dilators, with cross sectional geometry shown off to the side at various locations along the length of the guidewire. FIG. 17E shows a dilator having helical grooves.

FIG. 18A-18D show schematic side views of an exemplary catheter, illustrating a sequence for inverting a distal tip of the catheter and removing the guidewire from the catheter.

FIG. 19 shows a schematic side view of an exemplary catheter having a guidewire in a wall of the conduit of the catheter.

FIGS. 20A-20D show schematic side views of exemplary catheters in accordance with embodiments disclosed herein.

FIG. 21 is a partial schematic view of a catheter for use as an endo-tracheal (ET) tube.

FIG. 22 schematically shows an ET tube as disclosed herein inserted into a patient.

FIG. 23 depicts disassembly of a distal portion of an exemplary catheter in which the dilator and guidewire of the catheter are integrally formed.

FIG. 24A depicts a distal portion of an exemplary flexible catheter in an unbent configuration. FIG. 24B depicts the distal portion of the flexible catheter of FIG. 24A in a bent configuration.

FIGS. 25A-25H depict the sequential advancement, and retraction, of a guidewire and dilator within a conduit of an exemplary catheter as disclosed herein. FIG. 25I is a close-up perspective view of a distal end portion of the catheter following advancement of the guidewire and dilator.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, unless the context dictates otherwise, reference to “a portion” provides disclosure of embodiments in which only a single such portion is provided, as well as embodiments in which a plurality of such portions are provided.

As used herein “or” should be understood to be an inclusive or unless context dictates otherwise. For example, when separating items in a list, “or” should be interpreted as being inclusive, i.e., the inclusion of at least one, but also including more than one, of a number or list of elements, and, optionally, additional unlisted items. In other aspects, the term “or” can refer to only a single element of a list of elements.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, the term “subject,” “patient,” or “individual” can be used interchangeably and refer to any organism to which an electrical pulse of this invention may be applied or administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like. The term does not denote a particular age or sex.

As used herein, “proximal end portion” refers to a portion of a device that remains outside of the patient, and “distal end portion” refers to a portion of the device entering the patient. The distal end portion of a device can, thus, be understood to be an insertional portion of the device.

As used herein “inlet” and “outlet” when referring openings to the lumen, are not intended to be limiting. That is, fluid and objects are not required to travel from the inlet to the outlet. For example, the term “inlet” is used in referring to an opening of the lumen that is configured for positioning within a bladder. However, in some aspects, the “inlet” can serve as an “inlet/outlet.” For example, in some aspects, fluid can be delivered through the lumen from the “outlet” to the “inlet.”

Disclosed herein is a catheter that is configured for inserting into an interior passage of a patient. For example, in some aspects, the catheter can be a transurethral catheter for insertion into a urethra (e.g., to access a bladder). In other aspects, the catheter can be used for a vascular, gastrointestinal, biliary, or other interior passage. Accordingly, the catheter can be dimensioned for use in a particular interior passage. Further, in various aspects, the catheter can be configured (e.g., sized) for human subjects. In other aspects, the catheter can be configured for use with animal subjects.

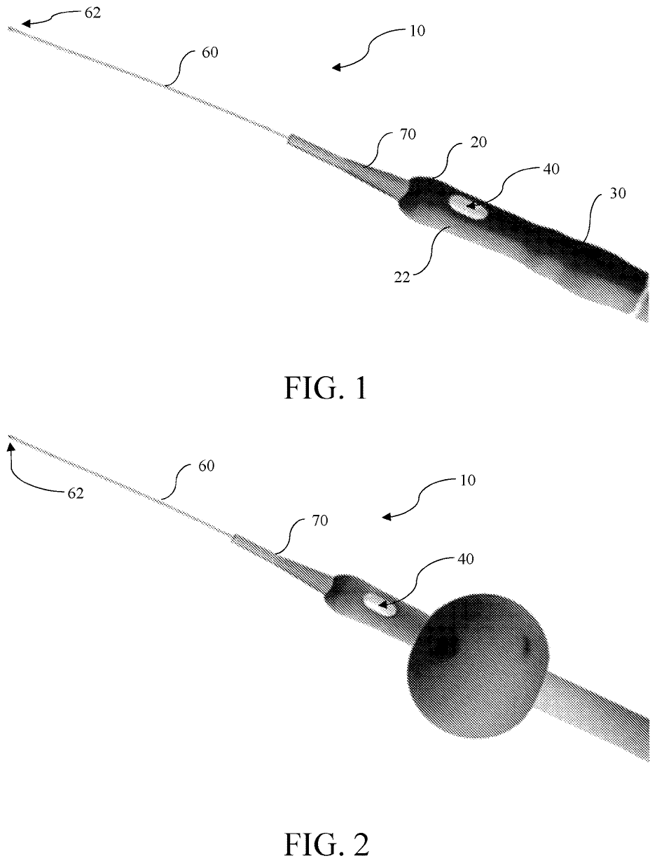

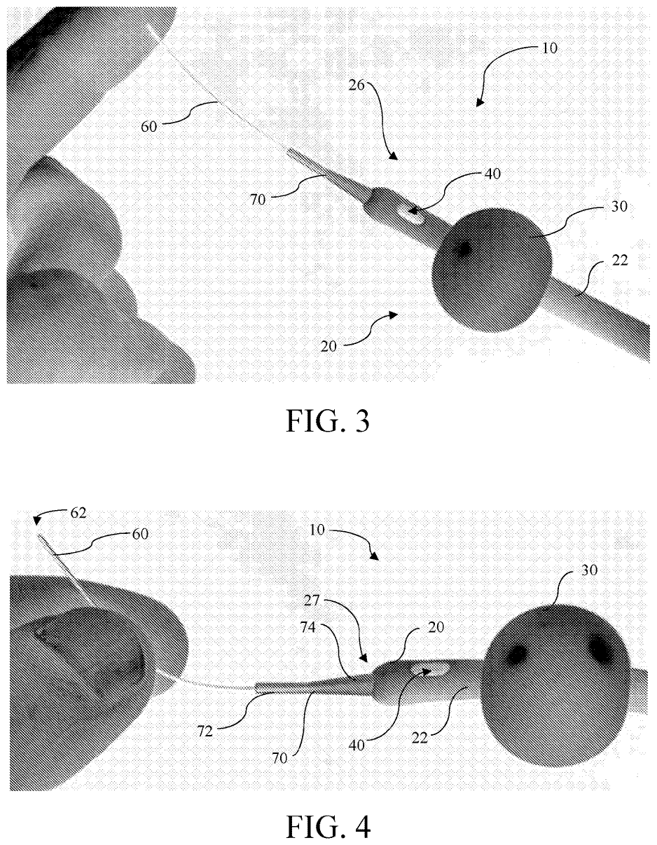

Disclosed herein, and with reference to FIGS. 1-6, is a catheter 10 having a longitudinal axis 12. The catheter 10 can comprise a conduit 22 having a proximal end portion 24, a distal end portion 26, and a lumen 28 that extends through the conduit 22 from the proximal end portion 24 to the distal end portion 26. The distal end portion 26 of the conduit 22 can comprise a distal tip 27 of the conduit 22.

A guidewire 60 can couple to the conduit 22 and can extend distally of the distal end portion of the conduit 22. The guidewire 60 can be flexible so as to not damage the urethra while sufficiently rigid to permit insertion into the urethra. In some aspects, the guidewire 60 can extend through the lumen 28 of the conduit 22. Referring also to FIG. 19, in other aspects, the guidewire 60 can be positioned within the wall of the conduit 22. In other aspects, the guidewire 60 can be positioned outside, and coupled to, the conduit 22. The guidewire 60 can provide stiffness and prevent buckling of the catheter 10 when the catheter is being advanced through an interior passage. The guidewire 60 can enhance the ability to access areas of the body (such as, for example, a bladder) beyond a stricture.

In some aspects, the catheter 10 can comprise a dilator 70 that is configured to facilitate opening (e.g., stretching or dilation) of a narrowing or stricture (e.g., a stenosis or a blockage) of an interior passage of a patient. More generally, the dilator can comprise an outer surface that serves as a transition from the cross sectional dimensions of the guidewire 60 to the cross sectional dimensions of the conduit 22. For example, in some aspects, the dilator can comprise a tapered structure as further described herein. In other aspects, the dilator 70 can be a balloon dilator that is configured to be inflated and deflated in order to open up a portion of the interior passage of the patient. Accordingly, in some aspects, a corresponding flow channel in communication with the dilator 70 can extend along the catheter 10. Said flow channel can receive fluid (e.g., air) for inflating the dilator. In some aspects, inflation of the dilator 70 can cause the dilator to expand radially outwardly. In some aspects, inflation of the dilator 70 can cause the dilator to expand distally and longitudinally. In still further aspects, inflation of the dilator 70 can cause the dilator to expand both radially outwardly and longitudinally distally. The dilator 70 can comprise a longitudinal opening that permits the guidewire to extend through the dilator (e.g., exiting through a hole at a distal tip of the dilator).

In some optional aspects, the guidewire 60 can be removably coupled to the conduit 22. In this way, once the catheter 10 is properly positioned in the urethra, the guidewire 60 can be removed. For example, the guidewire 60 can be pulled proximally from the conduit or from the braced tract alongside the conduit. In further aspects, the guidewire 60 can be pushed distally (e.g., pushed and pulled) in order to manipulate and access a strictured urethra or other interior passage of a patient. Thus, in some optional aspects, the guide wire can be configured for bidirectional movement. Optionally, the guidewire 60 can extend from the distal end portion 26 of the conduit 22 toward (and, optionally, beyond) the proximal end portion 24 sufficiently so that guidewire 60 can be gripped and pulled from the bladder once the catheter 10 is properly positioned in the urethra. In other aspects, a separate tether can couple to the guidewire, and the tether can extend toward (and, optionally, beyond) the proximal end portion 24 of the conduit 22 so that the tether can be gripped to pull the guidewire from the bladder once the distal end portion of the catheter 10 is properly positioned within the bladder. In some aspects, the guidewire 60 can be secured to a stable (e.g., a secure anchor) at the distal portion 26 of the conduit 22 to prevent the guidewire 60 from dislodging during insertion through the urethra and into the bladder. In other aspects, the dilator 70 and guidewire 60 can be coupled together, and the dilator and guidewire can be retracted through the conduit 22 only when sufficient force is applied to draw the dilator through a distal opening of the conduit 22. More generally, in some aspects, the dilator 70 can be advanced or retracted (e.g., deployed outwardly from or stay undeployed within the lumen of the distal end portion 26 of the conduit 22).

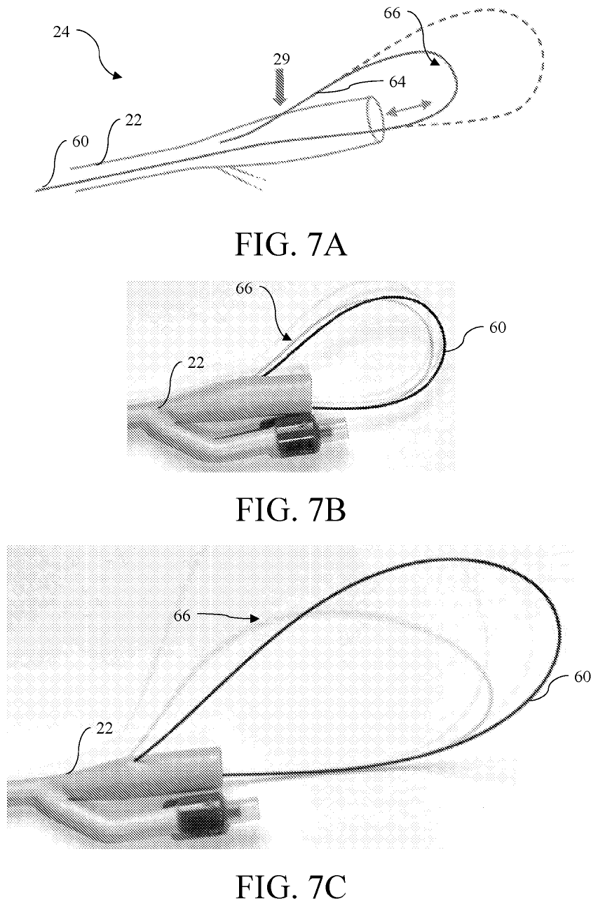

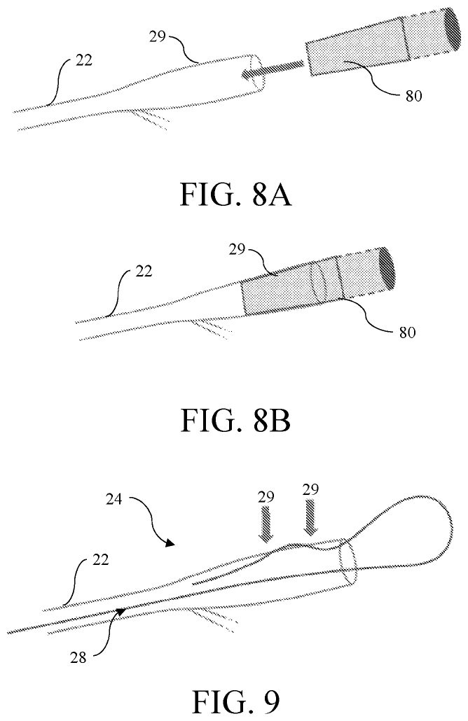

In some aspects, the guidewire 60 can be coupled to the conduit at the proximal portion 24 of the conduit 22. Referring to FIG. 7A-C, in some aspects, a proximal end portion 64 of the guidewire 60 can extend through the proximal end portion 24 of the conduit 22. In this way, the guidewire 60 can be fixedly coupled to the conduit 22 where the guidewire extends therethrough. The conduit 22 can define a hole 29 through which the guidewire extends. In some aspects, the hole can be oblique to the dimension of elongation of the conduit. The guidewire 60 can plug the hole 29 through the conduit so that the conduit does not leak. Further, referring also to FIGS. 8A-8B, the hole through which the guidewire 60 extends can be positioned sufficiently proximally so that when a connecting tube 80 of a urine bag is inserted into the conduit, the connecting tube 80 blocks the hole 29.

In some optional aspects, the guidewire 60 can form a loop 66. An operator (e.g., clinician or other member of a medical staff) can pull on the loop to draw the leading tip 62 of the guidewire 60 proximally through the conduit 22. Similarly, the operator can push on the wire to push slack from the loop through the conduit to advance the leading tip 62 distally.

Referring also to FIG. 9, in some optional aspects, the proximal end portion 64 of the guidewire 60 can extend through the proximal end portion 24 twice (e.g., starting from a proximal tip of the guidewire, the guidewire can extend through the wall of the conduit 22 from an interior of the conduit 22 to the exterior of the conduit and back through the wall of the conduit from the exterior to the interior of the conduit, within the lumen 28).

Referring to FIG. 10, in some optional aspects, a plug 90 can be inserted into the proximal end portion 24 of the conduit 22 with the guidewire 60 wedged between the conduit and the plug 90.

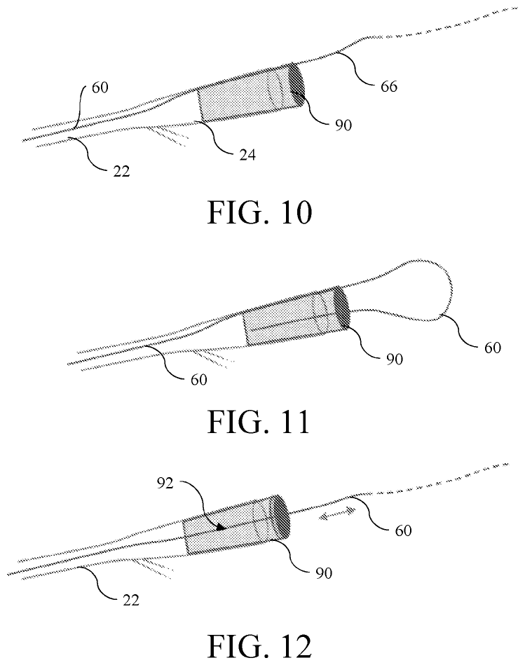

Referring to FIG. 11, in some optional aspects, the proximal end portion 64 of the guidewire 60 can be coupled to the plug 90. For example, the plug 90 can be formed around the guidewire 90. The guidewire 60 can optionally extend distally from the plug 90, form a loop 66, and then extend through the conduit 22. Referring to FIG. 12, in other aspects, the guidewire 60 can extend through a channel 92 of the plug 90. The channel 92 can have a sufficient cross section to permit the guidewire 60 to be pushed or pulled through the channel.

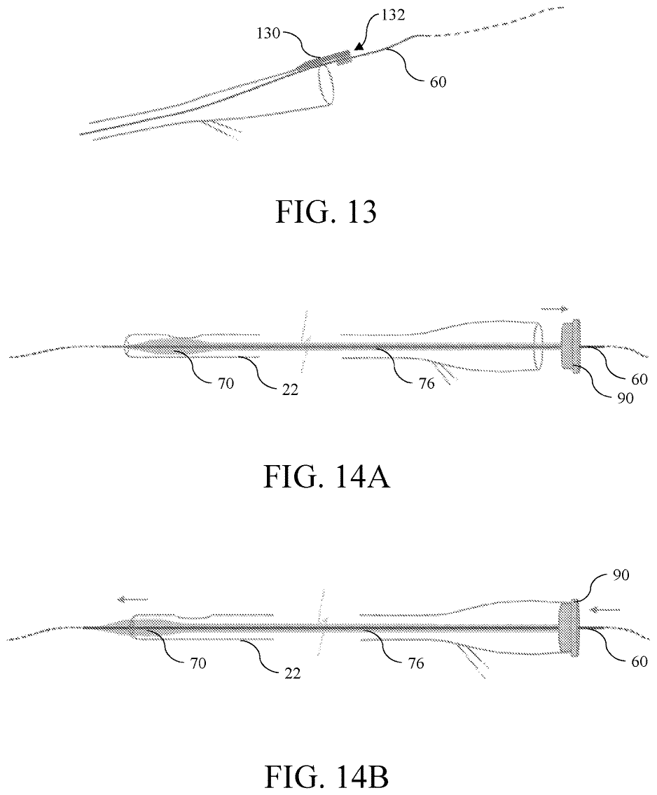

Referring to FIG. 13, in some aspects, an anchor can be coupled to the proximal end portion 24 of the conduit 22 for securing the guidewire 60 to the conduit 22. In some optional aspects, the anchor 130 can comprise an eye 132 through which the guidewire 60 can extend. In other aspects, the anchor 130 can comprise a clip, a tie, a tie and button, or any other suitable fastener for coupling the guidewire 60 to the conduit.

Referring to FIGS. 14A and 14B, in some aspects, the dilator 70 can comprise a body 76 that extends proximally along, and couples to, the guidewire 60. For example, the body 76 can circumferentially surround a portion of the guidewire 60. In some aspects, the dilator 70 can be overmolded on the guidewire 60.

The body 76 of the dilator 70 can provide structural support to the guidewire 60 so that the coupled guidewire 60 and body 76 have more rigidity than just the guidewire 60 itself. In this way, the guidewire 60 and dilator 70 can be more easily advanced through a stricture (e.g., a stenosis or a blockage) of the interior passage of a patient. As stated herein, in some aspects, the interior passage can be a urethra. In other aspects, the interior passage can be a vascular passage (e.g., blood vessels), a gastrointestinal tract, a biliary tract, an oral passage, a pharyngeal passage, a laryngeal passage, or a pulmonary tract, or other such passage. More generally, the vascular passage can be any passage for a human or animal patient. Accordingly, the disclosed catheter can be used for accessing any organ or lumen, and can be used in any corresponding medical subspecialty. Thus, it is contemplated that the interior passage can be any passage within the body of a patient that needs to be accessed via a catheter as disclosed herein. For example, as described herein, it is contemplated that the disclosed catheter 10 can be deployed within interior passages that need to be accessed during a pulmonary procedure, a cardiothoracic procedure, a neurosurgical procedure, an orthopedic procedure, an otolaryngological procedure, an obstetric/gynecological procedure, a reconstructive surgical procedure, a radiological procedure, an interventional radiological procedure, an anesthetic procedure, or combinations thereof.

In some aspects, the dilator 70 can couple to the plug 90. For example, in some optional aspects, the dilator 70 can be integral to the plug 90. The plug 90 can serve as a handle. The plug 90 can further limit proximal movement of the dilator 70 and guidewire 60.

Still referring to FIGS. 14A and 14B, in other aspects, the guidewire 60 and dilator 70 are separate structures so that the guidewire 60 can be advanced and retracted relative to the dilator 70.

In some aspects, the lumen 28 can have a length along the longitudinal axis 12, and the guidewire 60 can extend through the entire length of the lumen 28. In other aspects, the guidewire 60 can extend along only a portion of the length of the lumen 28. In some aspects, the guidewire 60 can extend into the conduit 22 but can remain entirely outside of the lumen 28. Optionally, the lumen 28 can be a first lumen, and the conduit 22 can define a second lumen that runs parallel to the first lumen, with the guidewire 60 received within the second lumen. In still further aspects, a braced tract can couple to, and extend along, the conduit 22, and the guidewire 60 can be received within the braced tract.

In alternative aspects, the guidewire 60 can be nonremovably coupled to the conduit 22. For example, the guidewire 60 can remain in the bladder (e.g., curled softly against inner walls of the bladder) for temporary purposes (e.g., 1-2 days). Optionally, after a determined duration to allow the natural urethral lumen to mature and establish patency (e.g., heal after the primary insertion/dilation), the catheter 10 can be removed. A standard Foley catheter can then be used to replace the catheter 10. That is, initially, the disclosed catheter can be used as a first catheter for preparing the urethra, and traditional Foley catheters can be used thereafter.

In aspects in which the guidewire 60 is removable, the guidewire can comprise a stop that inhibits further distal movement of the guidewire 60 relative to the conduit 22. In other aspects, the guidewire does not comprise a stop to permit an operator to select a distance that the guidewire protrudes distally from the distal tip 27 of the conduit 22.

The guidewire 60 can have a leading tip 62 opposite the distal end portion of the conduit. Optionally, the leading tip 62 of the guidewire 60 can be rounded.

In various aspects, the guidewire 60 can have a cross sectional outer dimension from about 0.45 mm (0.018 in) to about 1 mm (0.039 in). These dimensions can be advantageous for transurethral catheters. In other aspects, the cross-sectional outer dimension can be selected based on the interior passage for which the catheter is configured. For example, in some aspects, the cross sectional outer dimension can range from 0.5 mm to 2 mm. Optionally, the cross-sectional outer dimension of the guidewire can be a diameter. In other aspects, the guidewire need not have circular cross sections. For example, the guidewire 60 can have elliptical cross sections.

In some optional aspects, the guidewire 60 can comprise a coating. For example, the coating can be hydrophilic. A hydrophilic coating can comprise two compounds: polyethylene oxide (PEO) and polyvinylpyrrolidone (PVP). These hydrophilic compounds can be advantageously used because of their biocompatibility and ability to reduce friction between wire and urethral wall in order to maneuver through areas of tortuosity and stenoses. However, the more hydrophilic a wire is, the less tactile feedback the wire provides the operator and the more likely the wire can enter the subintimal space. This is important when confronted with complex lesions where the operator may require better tactile cues. Accordingly, in other aspects, the coating can comprise a hydrophobic coating. The hydrophobic coating can be silicone-based and can be less lubricious than hydrophilic coatings. The hydrophobic coating can provide improved tactile feedback and can be more likely to stay in the true lumen and thus less likely to cause wall perforations. For example, the hydrophobic coating can provide hydrophilic friction. In some aspects, the coating can vary along the length of the guidewire 60. For example, in some aspects, only a portion of the guidewire 60 is coated. In other aspects, different portions of the guidewire 60 can have different coatings (e.g., different coating materials) and/or different thicknesses of coating. In some aspects, the stiffness of the guidewire can be consistent along the length of the guidewire. In other aspects, the stiffness of the guidewire can vary along the length of the guidewire. For example, different materials (e.g., materials of different thicknesses) can be included along different lengths of the guidewire.

In exemplary aspects, the coating 60 can comprise one or more of the following: acid modified polyolefin (e.g., TAMFER™ coating), alpha-hydroxy ketone, antibacterial coating, antimicrobial, antiseptic, benzophenone, DEHP-free, ethylene, ethylene and propylene, ethylene based polymer (EBP), ethylene vinyl alcohol (EVAL), hydro-coat (Abbott vascular), HYDRO-TRACK® coating (provided by Medtronic), latex-free, Nobel metal, nylon elastomer, plastic (e.g., vinyl, or PVC), polyether block amide (PEBAX), polytetrafluoroethylene (PTFE), polyvinyl alcohol (PVOH), polyvinylpyrrolidone (PVP), PRO/PEL® coating (provided by Medtronic), polytetrafluroehylene (PTFE), red rubber latex, rubber, silicone, silicone-elastomere, silver, stainless steel, superhydrophobic material, or thermoplastic polyurethane (TPU).

In some optional aspects, the guidewire 60 can comprise a material that changes at least one physical property with temperature. For example, the guidewire can optionally comprise nitinol. The changing physical property can advantageously be used to manipulate stiffness during insertion. In some optional aspects, the guidewire 60 can comprise stainless steel. In some optional aspects, the guidewire 60 can comprise a dissolvable material. In this way, the guidewire 60 can be dissolved when received within the catheter. In exemplary aspects, the guidewire can comprise multiple components. For example, in some aspects, the guidewire can comprise a wire-wrapped stainless steel core. The wire-wrapped stainless steel core can optionally comprise a coating (e.g., PTFE). The guidewire can have a relatively stiff proximal end to enable advancement via pushing and a relatively flexible end to inhibit trauma during advancement.

In some exemplary aspects, the guidewire 60 can optionally be formed from HIWIRE wire, manufactured by Cook Medical (Bloomington, IN). However, it is contemplated that other commercially available guidewires can be used.

In some optional aspects, the catheter 10 can comprise a dilator 70 that extends distally from the distal end portion 26 of the conduit 22. At least a portion of the dilator 70 can have a taper in a distal direction. In some optional aspects, the dilator 70 can surround the guidewire 60 and can receive a portion of the guidewire therein. The taper of the dilator can minimize resistance and perforation relative to a catheter without a tapered dilator.

Optionally, the guidewire 60 can extend beyond the distal end portion 26 of the conduit 22 by a first length, and the dilator 70 can extend beyond the distal end portion of the conduit by a second length that is less than the first length.

The dilator 70 can have a length along the longitudinal axis 12 of the catheter 10. In some optional aspects, the dilator can have a continuous taper along the length of the dilator. In alternative aspects, the dilator 70 can comprise a distal end portion 72 having consistent cross sections (e.g., circular cross sections) in planes perpendicular to the length of the dilator 70. The dilator 70 can further comprise a proximal end portion 74 that defines the taper. The taper can optionally be a consistent taper (i.e., having a linear change in cross sectional dimension). For example, in some aspects, the dilator 70 can have a conical surface. In other aspects, the taper can be inconsistent along the length of the dilator. For example, optionally, the taper of the dilator 70 can have an increasing rate of tapering toward the proximal end.

The taper of the dilator 70 can be characterized as a change in diameter per unit of length along the dilator. For example, a change in diameter of 2 millimeters along a length of 2 mm can have a taper ratio of 1; a change in diameter of 1 millimeter along a length of 2 mm can have a taper ratio of 0.5. In some exemplary aspects, the taper can have a taper ratio from about 0.1 to about 2, or from about 0.2 to about 1. In some aspects, the taper of the dilator 70 can taper from greater than 2 mm to less than 1 mm.

In various aspects, the conduit 22 can have a cross sectional outer dimension (e.g., diameter) from about 1 mm to about 10 mm.

In some optional aspects, the conduit 22 can comprise rubber, silicone, polyurethane, or a combination thereof. In various aspects, the conduit 22 can comprise any suitable biologically compatible material.

Optionally, the catheter 10 can be an indwelling catheter. In other aspects, the catheter 10 can be a non-indwelling catheter.

Referring to FIGS. 1-6, in some optional aspects, the conduit 22 can be provided as a conduit assembly 20. The conduit assembly 20 can comprise the conduit 22 and a balloon 30 that is coupled to and configured to extend radially outwardly of the conduit 22. The balloon 30 can be configured to resiliently expand radially outwardly when a fluid is received therein. In some aspects, the fluid can be air or other gas. In some aspects, the fluid can be a biocompatible liquid. The balloon 30 can circumferentially, completely or partially extend around the conduit 22.

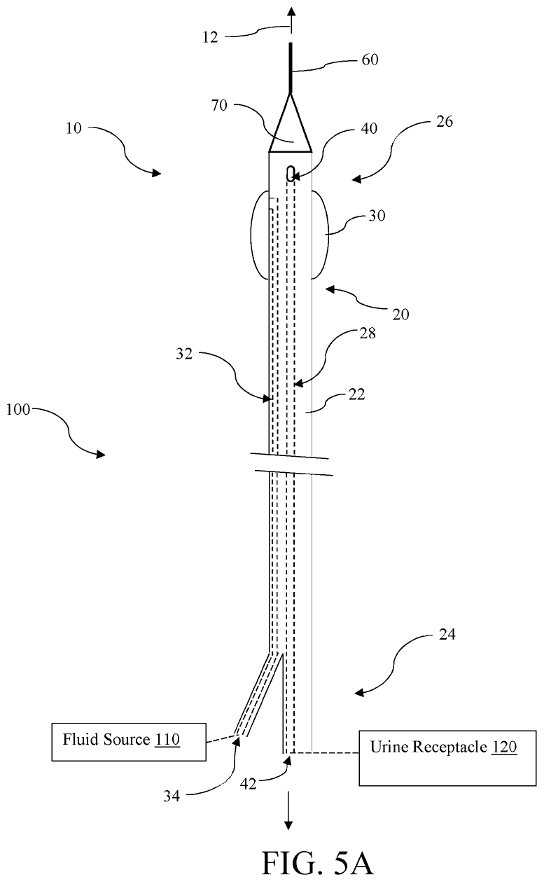

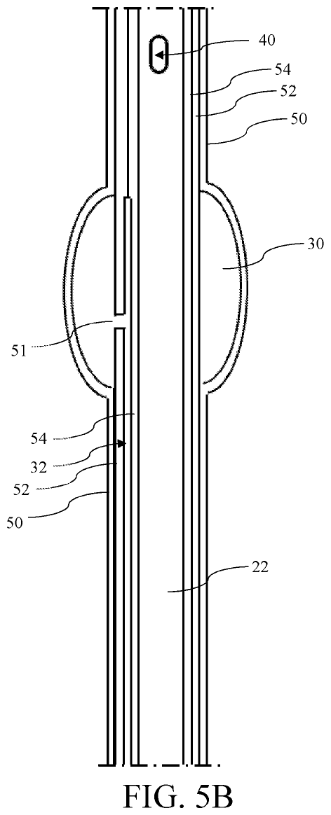

In exemplary aspects, the conduit assembly 20 can be configured in accordance with a conventional Foley catheter. For example, referring to FIG. 5B, the conduit assembly 20 can comprise an outer layer 50, an intermediate layer 52, and an inner layer 54. The balloon 30 can be defined by a portion of the outer layer 50 that is separated from the intermediate layer 52 and in communication with at least one opening 51 formed in the intermediate layer 52. The conduit assembly 20 can define a flow channel 32 between the inner layer and the intermediate layer for communicating fluid along the catheter 10 to and from the balloon 30 via the at least one opening 51.

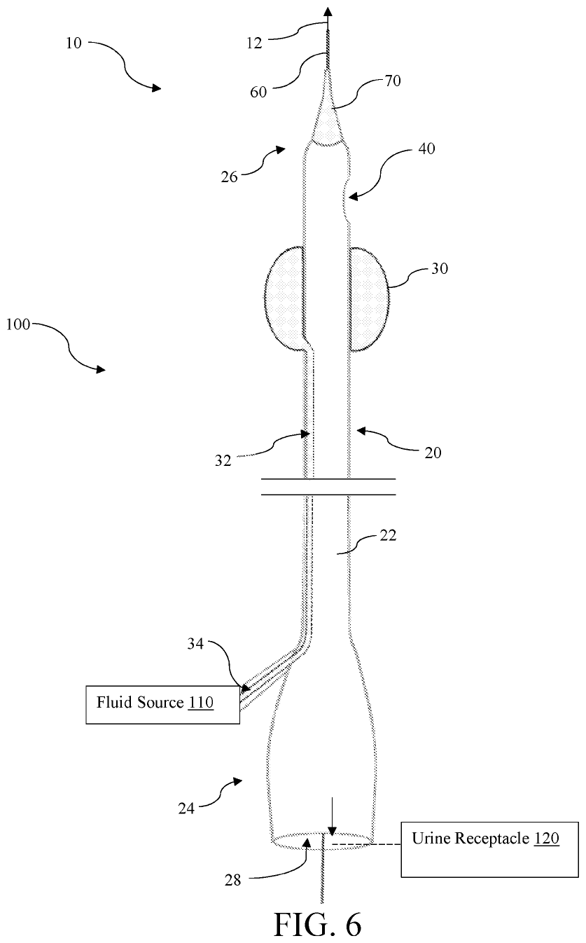

FIG. 6 shows another schematic drawing of the catheter 10. Although not necessarily show to scale or geometric shape, FIG. 6 indicates that the lumen 28 of the conduit 22 can be wide. The flow channel 32 can be incorporated within and extend along the wall of the conduit 22. The flow channel 32 can provide fluid communication from the proximal end portion 24 of the conduit 22 to the balloon 30 for inflating and deflating the balloon.

At least one inlet 40 to the lumen 28 can be positioned between the balloon 30 and the distal tip 27 of the conduit 22. For example, the conduit 22 can comprise two elongate inlets 40 on opposed sides of the conduit 22. In other optional aspects, the conduit 22 can omit radially extending inlets 40. In these aspects, the conduit 22 can comprise an inlet 40 only at the distal tip 27.

The catheter 10 can comprise a fluid inlet 34 in fluid communication with the balloon 30 via the fluid channel 32. The fluid inlet 34 can be configured to receive a fluid (e.g., liquid, gas, or other fluid) to inflate the balloon.

In some aspects, the catheter 10 can be a two-way catheter, having a lumen 28 and a flow channel 32. In further aspects, the catheter 10 can be a three-way catheter, having a flow channel 32, an inflow lumen, and an outflow lumen. In still further aspects, the catheter can be a four-way catheter.

In other aspects, the catheter 10 can be free of a balloon. That is, in these aspects, the catheter 10 does not comprise a balloon. For example, the catheter 10 can be a single lumen catheter. The single lumen catheter can be a non-indwelling catheter (e.g., a single-use catheter). Such a catheter can be used for self-catheterization, clean intermittent catheterization, emptying a bladder peri-operatively, or for instillation of intravesical medication.

Referring to FIG. 15A-15B, in some optional aspects, the guidewire 60 and the dilator 70 can be integrally formed. For example, in these aspects, the guidewire 60 and the dilator 70 can be unitarily formed as a monolithic component. In other aspects, the dilator 70 can be formed on the guidewire 60.

Referring to FIG. 16A-16B, in some aspects, the distal end portion 26 of the conduit 22 can comprise the dilator 70. For example, in some aspects, the distal end portion 26 of the conduit 22 can taper distally, and the distal taper can serve as the dilator 70. In some aspects, the distal taper can form an angle of less than 45 degrees, or less than 30 degrees, or from about 10 degrees to about 30 degrees with a central axis of the dilator. In some aspects, the angle of the taper can be consistent. In other aspects, the angle of the taper can change along the length of the dilator 70. In some aspects, the distal end portion 26 of the conduit can have any suitable profile, such as a helical profile, or have helical projections or grooves 78. In some optional aspects, and as illustrated in FIG. 16B and FIG. 27B, the distal end portion 26 of the conduit 22 can be flexible.

In some aspects, stiffness, hydrophilicity and other characteristics of the distal end portion 26 of the conduit 22 can be selected (e.g., by material selection) to optimize performance for a particular patient or indication. For example, the stiffness of the material can be greater than the other parts of the catheter in order to permit the catheter to be pushed through a stricture. As another example, the stiffness of the material can be less than the other parts of the catheter in order to inhibit trauma from the conduit 22.

In some aspects, the conduit 22 can have an outer coating. Optionally, the outer coating can be provided only on the distal end portion 26 of the conduit 22, or a different outer coating can be provided on the distal end portion 26 of the conduit 22.

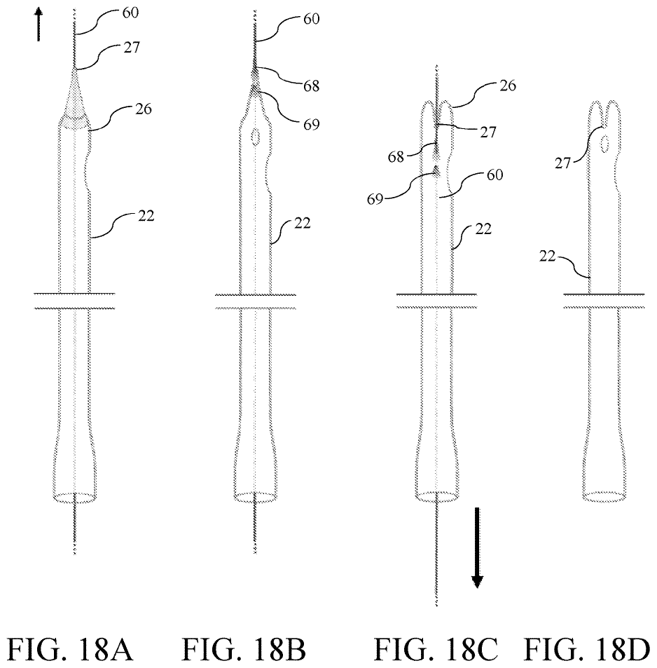

Referring to FIGS. 18A-18D, in some aspects, the distal end portion 26 of the conduit 22 (or a sub-portion thereof) can be inverted to reside within its lumen. In this way, the distal tip 27 of the conduit can be retracted within the conduit so that the distal-most end of the conduit is rendered rounded and smooth (and therefore non-traumatic) to prevent traumatic irrigation when indwelling in the urinary bladder. For example, a portion 68 of the guidewire distal of (beyond and outside of) the distal end portion 26 of the conduit 22 can have a larger cross sectional dimension than that of the distal end opening of the conduit 22. Accordingly, retraction of the guidewire can cause said larger portion 68 of the guidewire to catch on the distal end portion 26 of the conduit 22 to invert the distal end portion 26 within the lumen 28. In further aspects, the guidewire 60 can further be withdrawn from the conduit 22. For example, further retraction of the guidewire 60 can cause the distal end opening of the conduit 22 to resiliently expand to permit the larger portion of the guidewire to pass through. The guidewire 60 can further comprise a widened portion 69 that remains proximal of the distal tip 27 to inhibit undesired advancement of the guidewire.

In some aspects, the guidewire 60 can have a consistent outer diameter (or other major cross sectional dimension) along an entire length of the guidewire. In other aspects, the outer diameter (or other major cross sectional dimension) of the guidewire 60 can vary along the length of the guidewire. For example, in some aspects, the guidewire 60 can have a varying outer diameter (or other major cross sectional dimension) in order to vary the flexibility and the dilatational functionality of the guidewire 60 along particular sections.

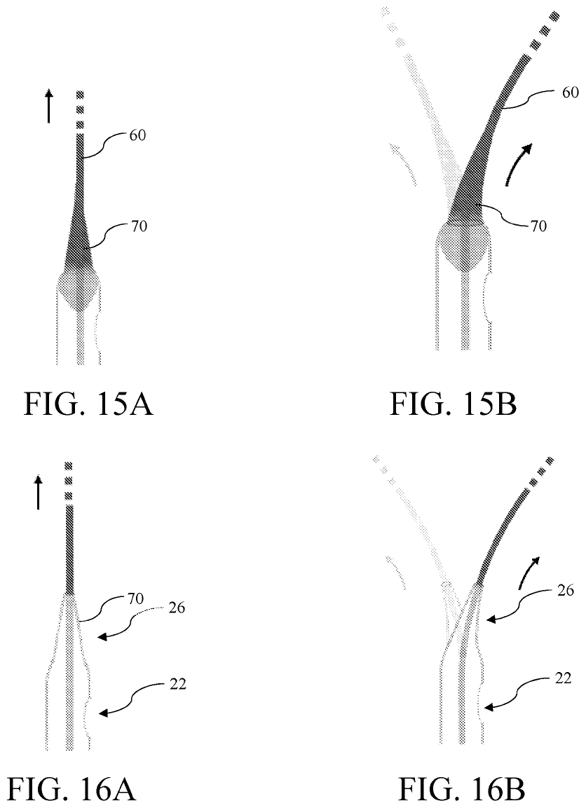

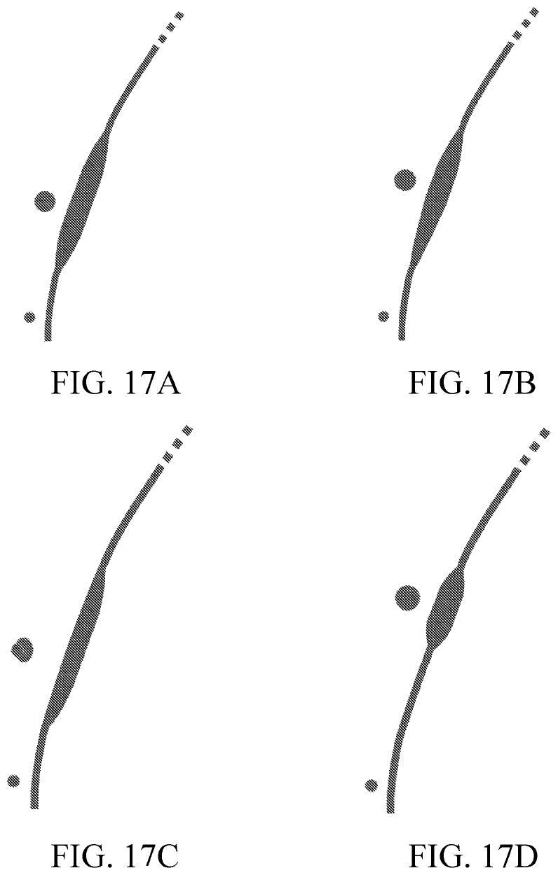

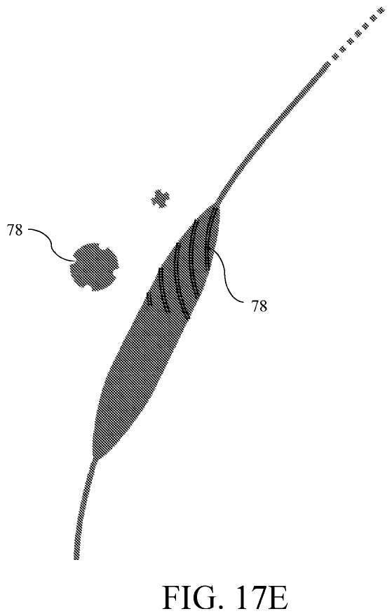

In some aspects, the guidewire 60 can have one or more surface features along the length of the guidewire. For example, the guidewire 60 can have a distal taper that either serves as the dilator 70 or is provided in addition to the dilator 70. In some aspects, the guidewire 60 can have a proximal taper. Said proximal taper can facilitate removal of the guidewire 60 from the catheter. In some aspects, the guidewire can have a helical shape. For example, the guidewire can be formed into a helical structure, or the outer surface can define a helical shape (e.g., having an auger-shape). In some aspects, guidewire 60 can have a plurality of surface features along its length. Optionally, in these aspects, the plurality of surface features can have the same length. In other optional aspects, the plurality of surface features can have different lengths. FIGS. 17A-17D illustrate different surface features that can be present along the length of the guidewire 60. For example, FIG. 17A illustrates a guidewire comprising a dilator that is generally symmetrical about a plane that is perpendicular to the longitudinal axis and bisects the dilator. FIG. 17B shows a dilator that has a more gradual proximal taper and a less gradual distal taper. FIG. 17C a guidewire comprising a dilator that is radially offset from a longitudinal axis that is centered within the guidewire. FIG. 17D shows a dilator that is shorter than the dilator of FIG. 17A. FIG. 17E shows a dilator 70 having helical grooves 78. Each dilator 70 can be advantageous for a different application. For example, certain internal passages or certain strictures can be traversed with different dilator geometries. Accordingly, in some aspects, a clinician can select the dilator based on the particular application. In these aspects, a kit can comprise a plurality of guidewires 60 having different dilators 70 (e.g., dilators of different sizes and/or geometries) integral or otherwise coupled thereto.

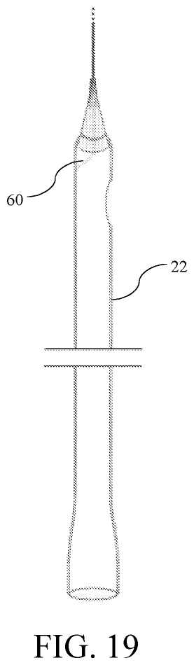

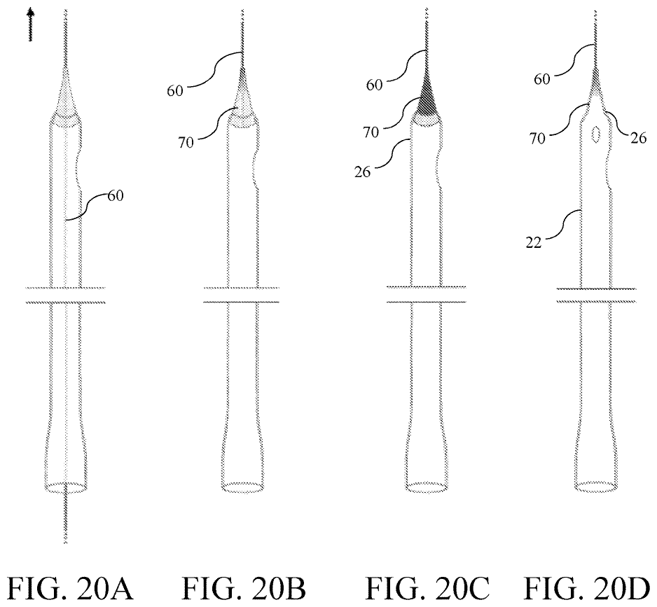

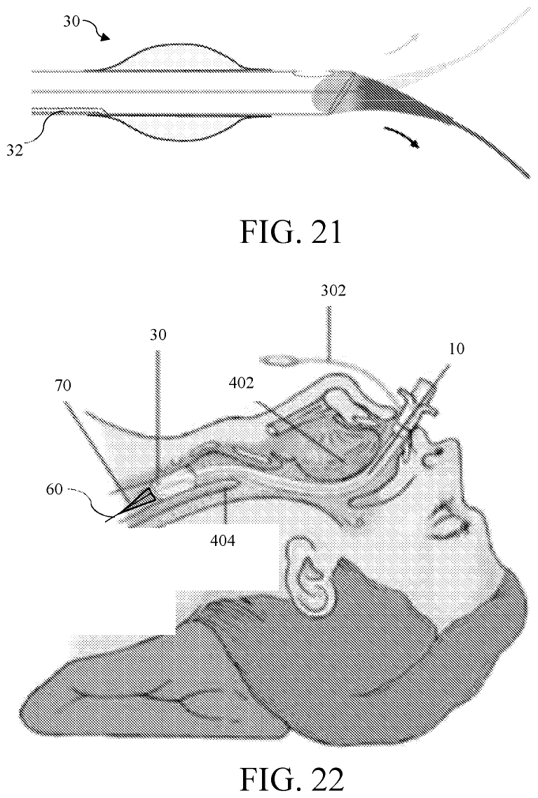

Referring to FIGS. 20A-20D, the conduit, guidewire, and dilator can be provided as separate components, or various combinations of these elements can be unitarily or integrally formed.

For example, referring to FIGS. 20A and 20B, the conduit 22, guidewire 60, and the dilator 70 can be separate elements, with the dilator being configured to be positioned distal of the conduit and configured to engage one or both of the conduit and the guidewire. Referring to FIG. 20C and FIG. 26, in some aspects, the dilator and guidewire can be integrally formed. Referring to FIG. 20D, in some aspects, the conduit 22 and dilator 70 can be integrally formed, optionally, as a unitary, monolithic component. In still further aspects, the conduit 22, dilator 70, and guidewire 60 can all be integrally formed. In each of these configurations, it is contemplated that the three components can cooperate to provide a graduated and/or gradual tapered profile.

Referring to FIGS. 20B-20D, in some aspects, the guidewire 60 need not extend proximally of the distal portion 26 of the conduit 22. Optionally, in these aspects, the guidewire 60 does not extend proximally of the dilator 70.

Referring to FIG. 5A, a system 100 can comprise a catheter 10 and a fluid source 110 in communication with the fluid inlet 34. In some aspects, an outlet 42 of the lumen 28 can be fluidly coupled to a urine collection receptacle 120.

A method can comprise inserting the distal end portion 26 of the conduit 22 into a urethra of a patient and inflating the balloon 30 to secure the distal end of the conduit within the bladder. Optionally, the method can comprise removing the guidewire 60 from the catheter 10 (e.g., withdrawing the guidewire from the conduit 22).

In various exemplary aspects, the disclosed catheter 10 can be used for self-catheterization, clean intermittent catheterization, emptying a bladder peri-operatively, or for instillation of intravesical medication.

In various aspects, the catheter 10 can be flexible along its entire length or along selected portions of its length in order to negotiate and accommodate curvatures and tortuosity within the interior passage or lumen where the catheter is positioned. For example, as shown in FIGS. 27A-27B, the guidewire, the dilator, and distal portions of the catheter can be sufficiently flexible to allow for bending to permit negotiation of curves within an interior passage. Optionally, the catheter 10 can be non-traumatic at all levels.

It is contemplated that the catheter 10 can be configured for its particular use. For example, the outer dimensions (e.g., diameter) can be selected for receipt into a particular interior passage of a patient. Further, the structure and materials used can be selected for the particular interior passage of the patient, as well as the mediums that are encountered within the interior passage. For example, a catheter can have a relatively more rigid stiffness for a gastrointestinal passage and a relatively less rigid stiffness for a urethra.

As further discussed herein, in addition to applications in urinary or gastrointestinal procedures, it is contemplated that the disclosed catheter 10 can be employed in a variety of interior passages within human and animal subjects. As one specific example, it is contemplated that during anesthesia, pulmonary and thoracic procedures, the disclosed catheter 10 can encounter air and be deployed within nasal/oral passages, the larynx, the pharynx, the trachea, the pulmonary tract, the bronchus, and/or the bronchioles of a subject. As another specific example, it is contemplated that during cardiothoracic/cardiovascular procedures, the disclosed catheter 10 can encounter blood, air, and/or lung tissue and be deployed within blood vessels. As another specific example, it is contemplated that during neurosurgical procedures, the disclosed catheter 10 can encounter spinal fluid, dura, muscle, and/or bone. As another specific example, it is contemplated that during orthopedic procedures, the disclosed catheter 10 can encounter synovial fluid, dura joint fluid, muscle, and/or bone. As another specific example, it is contemplated that during otolaryngological procedures, the disclosed catheter 10 can encounter air and be deployed within nasal passages, the larynx, the pharynx, the trachea, the pulmonary tract, the bronchus, and/or the bronchioles of a subject. As another specific example, it is contemplated that during obstetric/gynecological procedures, the disclosed catheter 10 can encounter air and/or blood and be deployed within the fallopian tubes, cervix, and/or uterus of a subject. As yet another specific example, it is contemplated that during radiological and interventional radiological procedures, the disclosed catheter 10 can encounter air, blood, urine, skin, fat, muscle, and/or facial tissue and be deployed within the gastrointestinal tract/lumen. As yet another specific example, it is contemplated that during anesthesia procedures, the disclosed catheter 10 can encounter air and be deployed within the trachea, pulmonary tract, bronchus, bronchioles, and/or blood vessels of a subject.

In some aspects, the catheter 10 can be an endo-tracheal (ET) tube. The catheter 10 can address challenges with endo-tracheal intubation for machine ventilation during anesthesia for surgical procedures or for artificial ventilation for several conditions/situations. The catheter 10 can further address difficulties due to anatomical and pathological constraints in accessing access larynx/trachea variety of reasons that include but not limited to size of patient, neck inflexibility, constrains in opening the mouth, etc.

Adverse events using conventional intubation means include, for example and without limitation: an inability to intubate; resorting to fiberoptics intubation; insertion of the ET tube into the wrong tube (e.g., esophagus); injury to the oral cavity, teeth, pharynx, larynx, vocal cords, and/or pulmonary tree (trachea); delays; vocal cord edema/inflammation; stridor; emergency tracheostomy; and/or death.

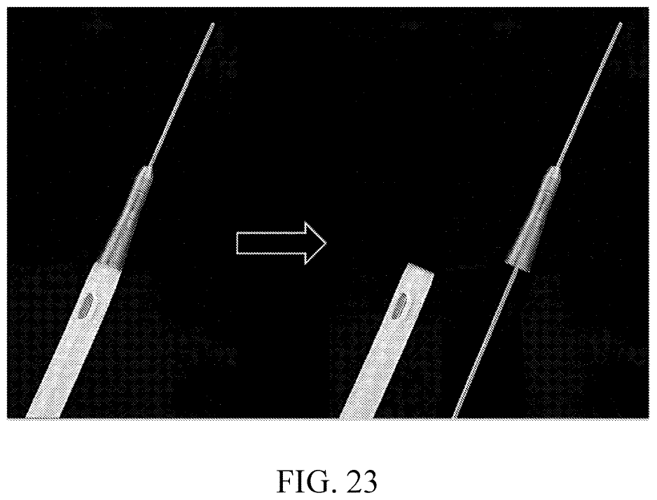

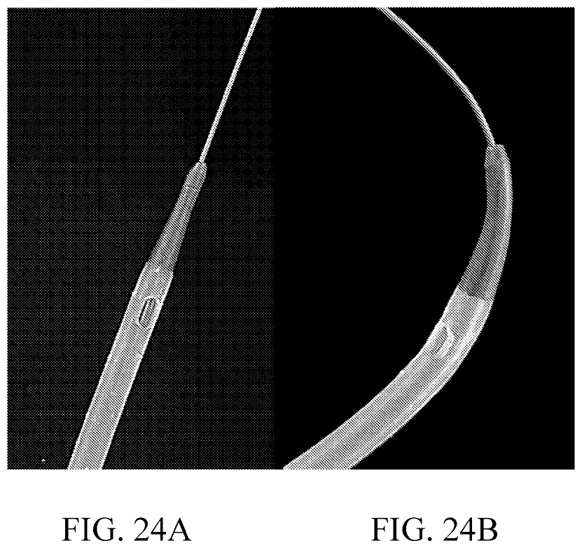

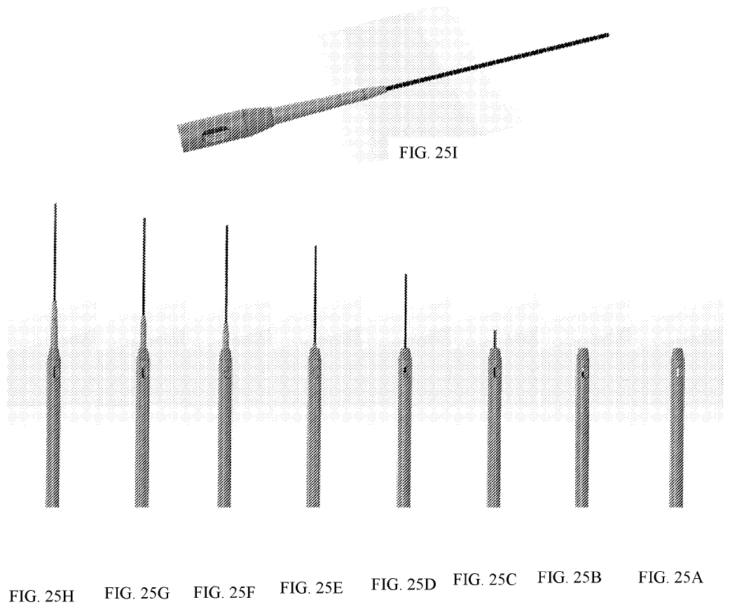

As further disclosed herein, and with reference to FIGS. 24-25, the catheter 10 can use a specialized non-traumatic guidewire that incorporates a smooth tapered portion, with a proximal wider part that fits snuggly within the distal end of the ET tube, and then tapers down to the same outer diameter of the guidewire. The guidewire and the tapered portion can be smooth, non-traumatic and malleable to negotiate curvatures and potentially provide access points through naturally restricted paths such as the tongue 402, the vocal cords 404. In exemplary aspects, the lumen of the catheter 10 can be wide, occupying most of the internal diameter of the catheter. Optionally, a separated flow channel 32 (e.g., a cuff conduit as depicted in dashed lines in FIG. 24) can be incorporated within and along a wall of the catheter. The cuff conduit 32 can fluidly connect the proximal part the catheter to a catheter cuff 302 for the purpose of inflating and deflating the ET tube cuff (balloon 30).

As described further herein, in some exemplary aspects, the guidewire and dilator can be unitarily formed.

In some optional aspects, the outer dimension (e.g., diameter) of the guidewire can be non-uniform along the length of the guidewire. In further aspects, the guidewire can comprise any suitable configuration (e.g., funnel, spiral, taper, etc.). Such configurations can be provided in single or multiple segments along the guidewire. For example, it is contemplated that a first segment of the guidewire can have a spiral profile/configuration, while a second segment of the guidewire can have a tapered profile/configuration. The configurations can optionally be of consistent lengths or of multiple different lengths along the guidewire.

Different materials can be used to determine the stiffness level of the guidewire and the characteristics (e.g., hydrophilic friction) of the outer coating. Stiffness and outer coating characteristics can be variable along the length of the guidewire, with the guidewire being malleable to allow for the desired curved configuration/shape during placement in individual patients. Optionally, the guidewire body within the ET tube can serve as a stylet.

In some aspects, and as illustrated in FIGS. 18A-18D, the guidewire and/or dilator can be removed out of the ET tube following successful placement of the tube. In some aspects, the catheter can have a conical tip that can be inverted into the distal tip upon pulling out the guidewire to render the catheter tip non-traumatic.

### EXEMPLARY AGENTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: a Catheter Comprising:

a conduit assembly having:
a conduit having a proximal end portion, a distal end portion, and a lumen that extends through the conduit from the proximal end portion to the distal end portion; and
a balloon coupled to and configured to extend radially outwardly of the conduit,
wherein the distal end portion of the conduit has:
   a distal tip of the conduit; and
   at least one inlet to the lumen that is positioned between the balloon and the distal tip of the conduit; and

a guidewire is coupled to the conduit, wherein the guidewire extends distally of the distal end portion of the conduit.

Aspect 2: The catheter of aspect 1, wherein the guidewire is removable from the lumen of the conduit.

Aspect 3: The catheter of aspect 1, wherein the guidewire is nonremovably coupled to the conduit.

Aspect 4: The catheter of any one of the preceding aspects, further comprising a dilator that extends distally from the distal end portion of the conduit, wherein at least a portion of the dilator has a taper in a distal direction, wherein the dilator surrounds and receives a portion of the guidewire.

Aspect 5: The catheter of aspect 4, wherein the guidewire extends beyond the distal end portion of the conduit by a first length, and wherein the dilator extends beyond the distal end portion of the conduit by a second length that is less than the first length.

Aspect 6: The catheter of aspect 4 or aspect 5, wherein the dilator has a length, wherein the dilator comprises:

a distal end portion having consistent cross sections in planes perpendicular to the length of the dilator; and

a proximal end portion that defines the taper.

Aspect 7: The catheter of any one of aspects 1-3, further comprising:

a dilator that extends distally from the distal end portion of the conduit; and

a flow channel in communication with the dilator,

wherein the dilator is configured to inflate upon receipt of fluid from the flow channel.

Aspect 8: The catheter of any one of the preceding aspects, wherein the guidewire has a cross sectional outer dimension from about 0.45 mm (0.018 in) to 1 mm (0.039 in).

Aspect 9: The catheter of aspect 8, wherein the cross-sectional outer dimension of the guidewire is a diameter.

Aspect 10: The catheter of any one of the preceding aspects, wherein the conduit has a cross sectional outer dimension from about 1 mm to about 10 mm.

Aspect 11: The catheter of aspect 10, wherein the cross-sectional outer dimension of the conduit is a diameter.

Aspect 12: The catheter of any one of the preceding aspects, wherein the guidewire comprises a coating.

Aspect 13: The catheter of aspect 12, wherein the coating is hydrophilic.

Aspect 14: The catheter of any one of the preceding aspects, wherein the conduit comprises rubber, silicone, polyurethane, or a combination thereof.

Aspect 15: The catheter of any one of the preceding aspects, wherein the catheter is an indwelling catheter.

Aspect 16: The catheter of any one of the preceding aspects, wherein the catheter is a non-indwelling catheter.

Aspect 17: The catheter of any one of the preceding aspects, wherein the guidewire has a leading tip opposite the distal end portion of the conduit, wherein the leading tip of the guidewire is rounded.

Aspect 18: The catheter of any one of the preceding aspects, wherein the guidewire comprises nitinol.

Aspect 19: The catheter of any one of the preceding aspects, wherein the least one inlet to the lumen comprises two elongate inlets on opposed sides of the conduit.

Aspect 20: The catheter of any one of aspects 5-7, wherein the dilator and the guidewire are integrally formed.

Aspect 21: The catheter of any one of aspects 1-3 or 8-20, wherein the distal end portion of the conduit comprises a taper or a helical shape that defines a dilator.

Aspect 22: The catheter of any one of the preceding aspects, wherein the guidewire has a length, wherein the guidewire defines a major cross sectional dimensions in cross section in respective planes perpendicular to the length, wherein the major cross sectional dimensions of the guidewire vary along the length of the guidewire.

Aspect 23: The catheter of any one of the preceding aspects, wherein the guidewire has a length, wherein the guidewire has a variable stiffness along the length of the guidewire.

Aspect 24: The catheter of aspect 12 or aspect 13, wherein the guidewire has a length, wherein the coating varies along the length of the guidewire.

Aspect 25: The catheter of any one of the preceding aspects, further comprising a fluid inlet in fluid communication with the balloon, wherein the fluid inlet is configured to receive a fluid to inflate the balloon.

Aspect 26: A system comprising:

a catheter as in aspect 25; and

a fluid source in communication with the fluid inlet.

Aspect 27: A method comprising:

inserting the distal end portion of the conduit of a catheter as in any one of aspects 1-25 into an interior passage of a patient; and

inflating the balloon to secure the conduit within the interior passage.

Aspect 28: The method of aspect 27, wherein the interior passage is a urethra.

Aspect 29: The method of aspect 27, wherein the interior passage is a blood vessel, a biliary tract, a gastrointestinal tract, an oral passage, a pharyngeal passage, a laryngeal passage, or a pulmonary tract.

Aspect 30: The method of aspect 27, further comprising removing the guidewire from the catheter.

Aspect 31: The method of any one of aspects 27-30, further comprising removing the distal end portion of the conduit of the catheter from the interior passage.

Aspect 32: The method of aspect 31, further comprising, prior to removing the distal end portion of the conduit of the catheter from the interior passage, inverting the distal end portion of the conduit.

Aspect 33: a catheter comprising:

a conduit having a proximal end portion, a distal end portion, and a lumen that extends through the conduit from the proximal end portion to the distal end portion, wherein the distal end portion of the conduit has:
a distal tip of the conduit; and
at least one inlet to the lumen; and

a guidewire is coupled to the conduit, wherein the guidewire extends distally of the distal end portion of the conduit.

Aspect 34: The catheter of aspect 33, further comprising a dilator having an outer surface, wherein the outer surface has a distal taper.

Aspect 35: The catheter of aspect 34, wherein the dilator is integral to the conduit.

Aspect 36: The catheter of aspect 34, wherein the dilator is integral to the guidewire.

Aspect 37: The catheter of aspect 34, wherein the dilator is integral to the conduit and the guidewire.

Those skilled in the art will recognize, or be able to ascertain using no more than routine experimentation, many equivalents to the specific embodiments of the method and compositions described herein. Such equivalents are intended to be encompassed by the following claims.

1. A catheter comprising:
a conduit assembly having:
a conduit having a proximal end portion, a distal end portion, and a lumen that extends through the conduit from the proximal end portion to the distal end portion; and
a balloon coupled to and configured to extend radially outwardly of the conduit,
wherein the distal end portion of the conduit has:
a distal tip of the conduit; and
at least one inlet to the lumen that is positioned between the balloon and the distal tip of the conduit; and
a guidewire is coupled to the conduit, wherein the guidewire extends distally of the distal end portion of the conduit.

2. The catheter of claim 1, wherein the guidewire is removable from the lumen of the conduit.

3. The catheter of claim 1, wherein the guidewire is nonremovably coupled to the conduit.

4. The catheter of claim 1, further comprising a dilator that extends distally from the distal end portion of the conduit, wherein at least a portion of the dilator has a taper in a distal direction, wherein the dilator surrounds and receives a portion of the guidewire.

5. The catheter of claim 4, wherein the guidewire extends beyond the distal end portion of the conduit by a first length, and wherein the dilator extends beyond the distal end portion of the conduit by a second length that is less than the first length.

6. The catheter of claim 4, wherein the dilator has a length, wherein the dilator comprises:
a distal end portion having consistent cross sections in planes perpendicular to the length of the dilator; and
a proximal end portion that defines the taper.

7. The catheter of claim 1, further comprising:
a dilator that extends distally from the distal end portion of the conduit; and
a flow channel in communication with the dilator,
wherein the dilator is configured to inflate upon receipt of fluid from the flow channel.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. The catheter of claim 1, wherein the least one inlet to the lumen comprises two elongate inlets on opposed sides of the conduit.

20. The catheter of claim 5, wherein the dilator and the guidewire are integrally formed.

21. The catheter of claim 1, wherein the distal end portion of the conduit comprises a taper or a helical shape that defines a dilator.

22. The catheter of claim 1, wherein the guidewire has a length, wherein the guidewire defines a major cross sectional dimensions in cross section in respective planes perpendicular to the length, wherein the major cross sectional dimensions of the guidewire vary along the length of the guidewire.

23. The catheter of claim 1, wherein the guidewire has a length, wherein the guidewire has a variable stiffness along the length of the guidewire.

24. The catheter of claim 1, wherein the guidewire has a length and a coating, wherein the coating varies along the length of the guidewire.

25. (canceled)

26. A system comprising:
a catheter as in claim 1, the catheter further comprising a fluid inlet in fluid communication with the balloon, wherein the fluid inlet is configured to receive a fluid to inflate the balloon; and
a fluid source in communication with the fluid inlet.

27. A method comprising:
inserting the distal end portion of the conduit of a catheter as in claim 1 into an interior passage of a patient; and
inflating the balloon to secure the conduit within the interior passage.

28. (canceled)

29. The method of claim 27, wherein the interior passage is a blood vessel, a biliary tract, a gastrointestinal tract, an oral passage, a pharyngeal passage, a laryngeal passage, or a pulmonary tract.

30. (canceled)

31. The method of claim 27, further comprising:
inverting the distal end portion of the conduit; and
after inverting the distal end portion of the conduit, removing the distal end portion of the conduit of the catheter from the interior passage.

32. (canceled)

33. A catheter comprising:
a conduit having a proximal end portion, a distal end portion, and a lumen that extends through the conduit from the proximal end portion to the distal end portion, wherein the distal end portion of the conduit has:
a distal tip of the conduit; and
at least one inlet to the lumen; and
a guidewire is coupled to the conduit, wherein the guidewire extends distally of the distal end portion of the conduit.

34. The catheter of claim 33, further comprising a dilator having an outer surface, wherein the outer surface has a distal taper.

35. The catheter of claim 34, wherein the dilator is integral to the conduit.

36. The catheter of claim 34, wherein the dilator is integral to the guidewire.

37. The catheter of claim 34, wherein the dilator is integral to the conduit and the guidewire.
